(19) United States
(12) Patent Application Publication
(10) Pub. No.: US 2022/0165394 A1
(43) Pub. Date: May 26, 2022

(54) METHOD, APPARATUS, AND NON-TRANSITORY COMPUTER READABLE MEDIUM FOR MANAGING MEDICAL IMAGES

(71) Applicant: HONGFUJIN PRECISION ELECTRONICS(TIANJIN)CO.,LTD., Tianjin (CN)

(72) Inventors: JUI-CHUAN CHEN, Neihu (TW); YU-TANG CHEN, New Taipei (TW)

(21) Appl. No.: 17/335,371

(22) Filed: Jun. 1, 2021

(30) Foreign Application Priority Data
Nov. 23, 2020 (CN) 202011325112.6

(51) Int. Cl. G16H 30/40; G06T 11/20; G16H 30/20; G06F 16/55; G16H 10/60

(52) U.S. Cl. CPC G16H 30/40; G06T 11/20; G16H 10/60; G06F 16/55; G16H 30/20

(57) ABSTRACT

A method for managing medical images implemented in an apparatus includes obtaining medical images; arranging identifiers to the medical images, wherein the identifiers are configured to identify individual patients; grouping the medical images according to the identifiers, each group of medical images corresponding to one individual patient; and displaying each group of the medical images according to a predetermined rule. An apparatus and a non-transitory computer readable medium for managing medical images are also disclosed.

### CROSS-REFERENCE TO RELATED APPLICATIONS

This application claims priority to Chinese Patent Application No.

202011325112.6 filed on Nov. 23, 2020, the contents of which are incorporated by reference herein.

### FIELD

The subject matter herein generally relates to medical image management, and particularly to a method, an apparatus, and a non-transitory computer readable medium for managing medical images.

### BACKGROUND

Nowadays, medical and biotechnology are more and more important. The use of medical images in medical data is increasing. There are a lot of medical image data categories and formats, such as CT, DR, CR, MR, ultrasound, and endoscopy, devices and apparatus for the medical images need to store a large amount of data. However, a system and a device for managing the data is needed for managing the medical images and their data from the large number of individual medical imaging devices.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a schematic view of an embodiment of an apparatus for managing medical images.

FIG. 2 is a schematic view of another embodiment of an apparatus for managing medical images.

FIG. 3 is a schematic view of an embodiment of a device for managing medical images applied in the apparatus shown in FIG. 1 or FIG. 2.

FIG. 4 illustrates a flowchart of an embodiment of a method for managing medical images executed by the device and apparatus.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” embodiment in this disclosure are not necessarily to the same embodiment, and such references mean “at least one.”

Furthermore, the term “module”, as used herein, refers to logic embodied in hardware or firmware, or to a collection of software instructions, written in a programming language, such as Java, C, or assembly. One or more software instructions in the modules can be embedded in firmware, such as in an EPROM. The modules described herein can be implemented as either software and/or hardware modules and can be stored in any type of non-transitory computer-readable medium or another storage device. Some non-limiting examples of non-transitory computer-readable media include CDs, DVDs, BLU-RAY, flash memory, and hard disk drives. The term “comprising” means “including, but not necessarily limited to”; it in detail indicates open-ended inclusion or membership in a so-described combination, group, series, and the like.

Nowadays, medical imaging technology is digitized. Medical imaging apparatus, such as apparatus of MR (magnetic), CT, ultrasound, X-ray, etc. are widely used in hospitals and physical examination organization. Picture Archiving and Communication System (PACS) is widely used for storing and managing digital images generated by the medical imaging apparatus to improve efficiency of inspecting and processing the medical images for the users.

The PACS used in the hospital is mainly for storing medical image data, such as data generated by apparatus of MR, CT, ultrasound, X-ray, infrared, microscopic, etc. through various of interfaces, such as virtual interface, DICOM, and network interface in a digital form. The stored medical image data can be rapidly reused under PACS authority, meanwhile the PACS provides some assistant diagnosis function according to the stored medical image data. Additionally, the PACS can be used to transmit the medical image data between several imaging apparatus. However, the PACS does not carry out any classifying and identifying of the medical image data for individual patients.

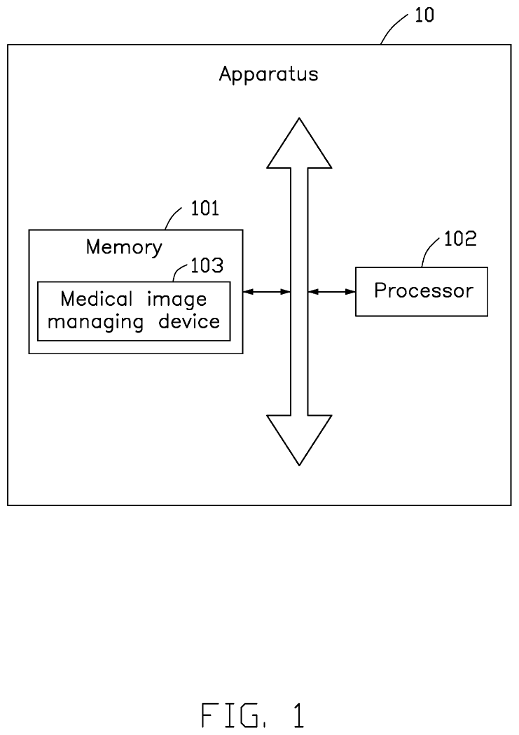

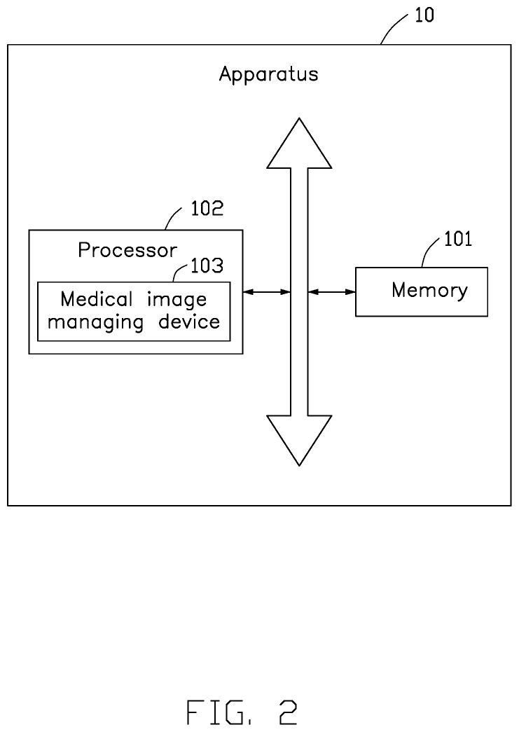

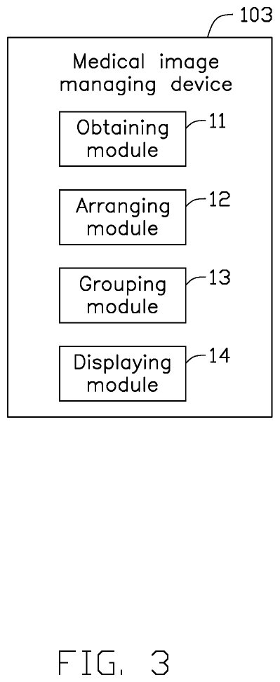

FIG. 1 illustrates an apparatus 10 including a memory 101, at least one processor 102, and a medical image managing device 103. The medical image managing device 103 can be stored in the memory 101 or the processor 102 (shown in FIG. 2) and processed by the processor 102. In at least one embodiment, the medical image managing device 103 can be non-transitory computer program medium stored in the memory 101 and processed by the least one processor 102 to perform a method for managing medical images and functions of modules of the medical image managing device 103 shown in FIG. 3. In at least one embodiment, the medical image managing device 103 can be non-transitory computer program medium integrated in the processor 102 and processed by the least one processor 102 to perform the method for managing medical images and functions of modules of the medical image managing device 103 shown in FIG. 3.

In at least one embodiment, the memory 101 can include various types of non-transitory computer-readable storage mediums. For example, the memory 101 can be an internal storage system, such as a flash memory, a random access memory (RAM) for the temporary storage of information, and/or a read-only memory (ROM) for permanent storage of information. The memory 101 can also be an external storage system, such as a hard disk, a storage card, or a data storage medium.

In at least one embodiment, the processor 102 can be a central processing unit (CPU), a microprocessor, a digital signal processor (DSP), an application specific integrated circuit (ASIC), a field-programmable gate array (FPGA), a data processor chip, a programmable logic device (PLD), a discrete gate/transistor logic device, or a discrete hardware component. The processor 102 may be electrically connected to other elements of the apparatus 10 through interfaces or a bus. In at least one embodiment, the apparatus 10 includes a plurality of interfaces configured to communicate with other medical apparatus and receive medical image data from the medical apparatus.

FIG. 3 illustrates the medical image managing device 103 including an obtaining module 11, an arranging module 12, a grouping module 13, and a displaying module 14.

The obtaining module 11 is configured to obtain medical image data. In at least one embodiment, the medical image data includes medical images generated by the medical apparatus.

The arranging module 12 is configured to arrange identifiers to the medical images. In at least one embodiment, the identifiers are configured to identify individual patients.

The grouping module 13 is configured to group the medical images according to the identifiers, thus each group of medical images is corresponding to one individual patient.

The displaying module 14 is configured to display the medical images according to a predetermined rule.

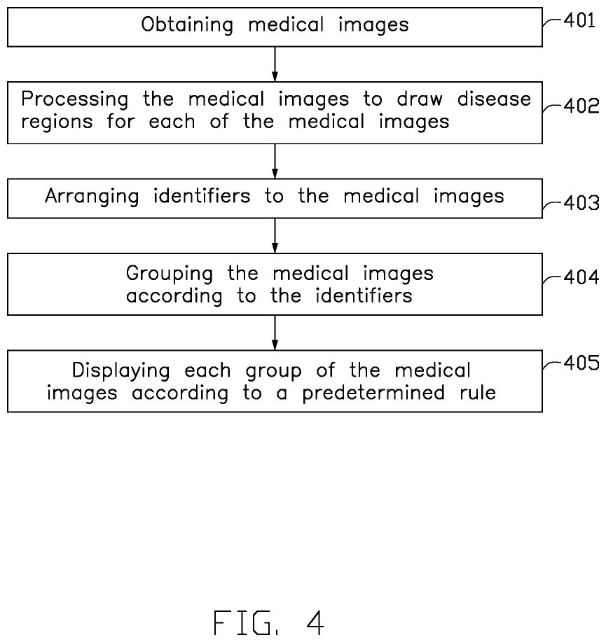

FIG. 4 illustrates a flowchart of an embodiment of a method for managing medical images. The method is provided by way of example, as there are a variety of ways to carry out the method. Each block shown in FIG. 4 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block 401.

At block 401, obtaining medical images.

In at least one embodiment, various medical images, generated by medical imaging apparatus such as MR, CT, ultrasound, X-ray, microscopy, etc., are stored in a digital form through interfaces such as virtual interface, DICOM, and network interface.

In at least one embodiment, the medical images can be uploaded by user interface of the apparatus 10, through connected interfaces, or/and downloading from a server to which the medical imaging apparatus has been uploading.

In at least one embodiment, the obtained medical images may be from different medical imaging apparatus, different timings, and for different individual patients.

At block 402, processing the medical images to draw disease regions for each of the medical images.

In at least one embodiment, the disease regions in the medical images, can be drawn through processing the medical images by a neural network system, to indicate areas where disease may exist.

In detail, predetermined parameters, input and/or preset by user, are configured to identify areas where disease may exist in each of the medical images. The medical images are processed according to the predetermined parameters, thus the disease regions in each of the medical images are drawn corresponding to the predetermined parameters.

In at least one embodiment, a range of predetermined parameters corresponding to disease A can be input and/or preset by user through user interface, then a data processor may generate a task and calculate the task. The data processor can be the neural network system. A progress of the task can be monitored by the user though the user interface.

In at least one embodiment, after obtaining the disease regions of each of the medical images, the data processor further evaluates an incidence rate of each of the disease regions.

In at least one embodiment, a predetermined time value can be input and/or preset by user through user interface and the medical images processed according to the predetermined time value. For instance, the predetermined time value can be a day or a week, processing the medical images everyday or every week for example, then the medical images can be in a time sequence, as day 1, day 2, day 3, etc.

At block 403, arranging identifiers to the medical images. In at least one embodiment, the identifiers are configured to identify individual patients.

In at least one embodiment, obtaining identity information, including name, gender, etc., relating to each of the medical images, assigning one identifier to each individual patient. The identifier is configured to indicate a relation between the medical image and the individual patient.

In at least one embodiment, when obtaining the medical images, arranging a corresponding identifier according to the identity information input by the user; when identity information of the medical images exists, assigning corresponding identifiers to the newly obtained medical images. In other embodiments, when obtaining medical images from medical imaging apparatus of hospital, obtaining identity information corresponding to the medical images from the medical system of the hospital. When assigning the identifiers to the medical images, binding the corresponding medical images to one identifier, or storing the corresponding medical images to a path corresponding to the identifier. Therefore, each of the medical images is bind to an identifier corresponding to an individual patient.

At block 404, grouping the medical images according to the identifiers, thus each group of medical images is corresponding to one individual patient.

In at least one embodiment, each of the identifiers is unique, when an identifier exists, meanwhile corresponding identify information of the individual patient and medical images exist. Individual patients corresponding to each of the medical images can be identified according to the corresponding identifiers, thus, grouping the medical images according to the identifiers, and each group of the medical images is corresponding to one individual patient. That is, medical images corresponding to a same identifier are in a same group.

At block 405, displaying each group of the medical images according to a predetermined rule.

In at least one embodiment, the predetermined rule can be a time sequence, displaying each group of the medical images in a time sequence, thus, the medical images of the same individual patient in different time period may be easily observed by the user, helping the user to observe a change of the disease. In other embodiments, the predetermined rule can be a sort sequence, displaying the medical images according to same sort of disease, that is, the medical images of a same sort can be displayed together, and medical images of different sorts are displayed sequentially.

In at least one embodiment, when displaying each group of the medical images in the predetermined rule, obtaining parameter data of the medical images, and displaying the parameter data together with the medical images. In at least one embodiment, the parameter data of the medical images may include image information and information of the disease regions. The parameter data of the medical images can be displayed in text.

Therefore, the medical images, the disease regions of the medical images, incidence rate of each of the disease regions, and the parameter data of the medical images can be provided to the user. Furthermore, the parameter data of the medical images can be statistically formed as charts and provided to the user. In at least one embodiment, the information of the disease regions in the parameter data can be statistically formed as a timeline chart, showing that a disease region data change in a time sequence.

A non-transitory computer-readable storage medium including program instructions for causing the apparatus 10 to perform the method for managing medical images is also disclosed.

For large numbers of medical images, the method for managing medical images may effectively manage individual patient data for tracking medical images and corresponding information in different time periods and provide the medical images from different medical imaging apparatus. The method for managing medical images also provides charts or text descriptions of disease regions of the medical images to the user for observing a change of the disease, thus, a diagnose effective can be improved.

It is believed that the present embodiments and their advantages will be understood from the foregoing description, and it will be apparent that various changes may be made thereto without departing from the spirit and scope of the disclosure or sacrificing all of its material advantages, the examples hereinbefore described merely being embodiments of the present disclosure.

What is claimed is:

1. A method for managing medical images implemented in an apparatus comprising:
obtaining medical images;
arranging identifiers to the medical images, wherein the identifiers are configured to identify individual patients;
grouping the medical images according to the identifiers, each group of medical images corresponding to one individual patient; and
displaying each group of the medical images according to a predetermined rule.

2. The method according to claim 1, further comprising:
drawing disease regions in each of the medical images.

3. The method according to claim 2, wherein drawing disease regions in each of the medical images comprises:
processing the medical images, according to predetermined parameters, to draw the disease regions corresponding to the predetermined parameters in the medical images.

4. The method according to claim 1, wherein displaying each group of the medical images according to a predetermined rule comprising:
displaying each group of the medical images in a time sequence.

5. The method according to claim 4, further comprises:
obtaining parameter data of the medical images; and
displaying the parameter data together with the medical images.

6. The method according to claim 5, wherein the parameter data of the medical images comprises image information and information of the disease regions, and the parameter data of the medical images is displayed in text.

7. The method according to claim 6, further comprises:
statistically forming the parameter date of the medical images as charts; and
displaying the parameter data of the medical images in charts.

8. The method according to claim 7, wherein the information of the disease regions in the parameter data is statistically formed as a timeline chart.

9. The method according to claim 2, further comprises:
obtaining a predetermined time value; and
processing the medical images termly according to the predetermined time value.

10. An apparatus for managing medical images comprising:
at least one processor; and
at least one memory coupled to the at least one processor and storing program instructions; the memory and the program instructions configured to, with the at least one processor, cause the apparatus to perform:
obtaining medical images;
arranging identifiers to the medical images, wherein the identifiers are configured to identify individual patients;
grouping the medical images according to the identifiers, each group of medical images corresponding to one individual patient; and
displaying each group of the medical images according to a predetermined rule.

11. The apparatus according to claim 10, wherein the memory and the program instructions configured to, with the at least one processor, cause the apparatus further to perform:
drawing disease regions in each of the medical images.

12. The apparatus according to claim 11, wherein drawing disease regions in each of the medical images comprises:
processing the medical images, according to predetermined parameters, to draw the disease regions corresponding to the predetermined parameters in the medical images.

13. The apparatus according to claim 10, wherein displaying each group of the medical images according to a predetermined rule comprising:
displaying each group of the medical images in a time sequence.

14. The apparatus according to claim 13, wherein the memory and the program instructions configured to, with the at least one processor, cause the apparatus further to perform:
obtaining parameter data of the medical images; and
displaying the parameter data together with the medical images.

15. The apparatus according to claim 14, wherein the parameter data of the medical images comprises image information and information of the disease regions, and the parameter data of the medical images is displayed in text.

16. The apparatus according to claim 15, wherein the memory and the program instructions configured to, with the at least one processor, cause the apparatus further to perform:
statistically forming the parameter date of the medical images as charts; and
displaying the parameter data of the medical images in charts.

17. The apparatus according to claim 16, wherein the information of the disease regions in the parameter data is statistically formed as a timeline chart.

18. The apparatus according to claim 11, wherein the memory and the program instructions configured to, with the at least one processor, cause the apparatus further to perform:
obtaining a predetermined time value; and
processing the medical images termly according to the predetermined time value.

19. A non-transitory computer readable medium comprising program instructions for causing an apparatus to perform at least the follow:
obtaining medical images;
arranging identifiers to the medical images, wherein the identifiers are configured to identify individual patients;
grouping the medical images according to the identifiers, each group of medical images corresponding to one individual patient; and
displaying each group of the medical images according to a predetermined rule.
